(19) United States
(12) Patent Application Publication
(10) Pub. No.: US 2002/0188342 A1
(43) Pub. Date: Dec. 12, 2002

(54) Short-term bioresorbable stents

(76) Inventors: Robert L. Rykhus JR., Edina, MN (US); Balkrishna S. Jadhav, Minnetonka, MN (US); Robert C. Grant, New Hope, MN (US)

Correspondence Address: OPPENHEIMER WOLFF & DONNELLY LLP, 840 NEWPORT CENTER DRIVE, SUITE 700, NEWPORT BEACH, CA, 92660, US

(21) Appl. No.: 09/920,871

(22) Filed: Aug. 2, 2001

Related U.S. Application Data
(60) Provisional application No. 60/295,298, filed on Jun. 1, 2001.

(51) Int. Cl. A61F 2/06

(52) U.S. Cl. 623/1.2; 623/1.22; 264/103; 219/121.71

(57) ABSTRACT

Bioresorbable, self-expanding stents intended to relieve stenoses occurring in organs, vessels, or other luminal structures within the human body are provided. The bioresorbable, self-expanding stents are composed of bioresorbable, biocompatible polymers. The bioresorbable stents provide openings that allow for tissue in-growth through the stent thereby fixing the stent in place and allowing the stent to be controllably degraded and excreted by the body.

### CROSS REFERENCE TO RELATED APPLICATIONS

This application claims the benefit of U.S. Provisional Application No. 60/295,298, filed Jun. 1, 2001, and whose entire contents are hereby incorporated by reference.

### BACKGROUND OF THE INVENTION

The present invention relates to stents and, in particular, to bioresorbable stents with short-term use applications. More specifically, the present invention relates to bioresorbable stents used in the treatment of urethral stenoses.

Stenosis is the narrowing of a lumen or opening. Stenosis occurs in organs, vessels, or other luminal structures within the human body. Stenosis resulting from disease or injury is often treated by surgical procedures. Conventional surgical techniques, however, may only offer temporary or partial relief as restenosis (recurrent stenosis) may develop. Thus, alternatives to surgical treatment of stenosis that provide luminal patency have been sought.

One approach for providing relief for stenosis has been the implantation of stents. Stents are mechanical scaffoldings that are inserted into the narrowed region of a lumen to provide and maintain patency. Traditionally, stents are made from metallic materials such as 316 stainless steel, MP35N alloy, superelastic Nitinol nickel-titanium, titanium alloys, and other alloys such as a wrought Cobalt-Chromium-Nickel-Molybdenum-Iron alloy. Recent developments, however, have led to stents made from bioresorbable polymers. Representative bioresorbable polymers include polyanhydrides, polycaprolactones, polyglycolic acids, poly-L-lactic acids, poly-D-L-lactic acids, and polyphosphate esters.

The development of stents for use in medical procedures has been a major advance in treating narrowed lumens; however, a variety of complications can and do occur with in vivo stent delivery and/or deployment. Complications such as restenosis caused by excess epithelialization or stent encrustation may result from long-term stent depolyment. Accordingly, surgical removal of a stent may become necessary. Moreover, removal of the stent becomes necessary where stents are used for short-term applications. Thus, there was a need to provide for reliable and non-invasive removal of stents.

A variety of minimally invasive products and procedures have been developed to provide reliable and efficient stent removal. Devices and/or assemblies allowing for an extraction of a stent are known and include, for example, United States Patent Number (USPN) U.S. Pat. No. 5,474,563, U.S. Pat. No. 5,624,450 and U.S. Pat. No. 5,411,507. While these removal systems are effective and safe to the patient, they have the disadvantage of being complicated to use and require direct surgeon involvement.

Recently, stents made from bioresorbable, biocompatible materials have been developed to dispense with complicated and potentially invasive stent removal procedures. These bioresorbable stents eliminate removal procedures because they gradually hydrolyze in the body. Stent fragments may then be excreted, as in the case of urethral and bowel stents, or the nontoxic soluble degradation products may be absorbed and metabolized. Stents comprised of bioresorbable materials are known and include, for example, U.S. Pat. No. 5,670,161, U.S. Pat. No. 5,085,629, U.S. Pat. No. 5,160,341, and U.S. Pat. No. 5,441,515.

Given the advancements in stent technology, however, there remains a need for bioresorbable stents that provide enough radial strength to maintain luminal patency. Furthermore, there is also a need to have bioresorbable stents that have controlled degradation without total stent collapse and resulting obstruction. Moreover, there is a need for cost-effective biocompatible stents and processes for making stents that have differing functional lives.

Therefore, it is an object of the present invention to provide a bioresorbable stent with large radial forces to alleviate stenoses.

It is yet another object of the present invention to provide a bioresorbable stent that provides controlled degradation.

### BRIEF SUMMARY OF THE INVENTION

These and other objectives not specifically enumerated here are addressed by a bioresorbable stent and associated methods which provide a bioresorbable stent having controlled stent degradation and excretion or resorption over a period of time thereby preventing total stent collapse and obstruction.

One embodiment made in accordance with the teachings of the present invention relates to bioresorbable stents comprising cylindrical sleeves having first ends and second ends. A latticed network formed from a plurality of monofilaments having an alternating braiding pattern is, disposed between the first end and the second end of the cylindrical sleeve. The monofilaments of this embodiment are spaced apart thereby forming openings between the monofilaments. These openings allow tissue in-growth and fixation of the individual monofilaments of the stent thereby fixing the stent in place and allowing for controlled degradation without total stent collapse and obstruction. Moreover, bioresorbable, self-expanding stents made in accordance with the teachings of the present invention provide large radial forces that maintain the patency of the occupied lumen. Furthermore, bioresorbable stents may be annealed and irradiated according to manufacturer defined parameters resulting in stents having variable, in vivo functional lives, The in vivo functional life of a bioresorbable stent is defined as the minimum length of time that the implanted stent would maintain adequate physical integrity and strength to maintain patency of a constricted region of a body lumen.

In another embodiment of the present invention, bioresorbable stents are formed by injection molding process or an extrusion process. The bioresorbable stents comprise tubular sheaths having first ends and second ends. The tubular sheath also contains fenestrations formed in the tubular sheath. The fenestrations of this particular embodiment provide openings in the stents that allow for tissue in-growth through the stents thereby fixing the stents in place and allowing the stents to be controllably degraded and excreted or absorbed by the body.

The present invention also provides methods for producing the bioresorbable, self-expanding stents of the present invention. A first method includes the steps of providing a plurality of biocompatible, bioresorbable monofilaments, braiding the monofilaments into a latticed network, annealing and irradiating the latticed network to achieve a predetermined in vivo functional life. A second method includes the steps of injection molding or extruding a bioresorbable polymer into a tubular sheath, cutting fenestrations into the tubular sheath, and annealing the tubular sheath to achieve a predetermined in vivo functional life.

Additional objects and advantages of the present invention will become readily apparent to those skilled in the art from the following detailed description, wherein only the preferred embodiments are shown and described, simply by way of illustration of the best mode contemplated of carrying out the invention. It is also contemplated that the present invention is capable of modification in various respects, all without departing from the scope and spirit of the present invention. Accordingly, the drawings and description are illustrative and not intended to be a limitation thereof.

### BRIEF DESCRIPTION OF THE DRAWINGS

FIG. 1A is a side view of the bioresorbable stent made in accordance with the teachings of the present invention.

FIG. 1B is an end view of the bioresorbable stent made in accordance with the teachings of the present invention.

FIG. 1C is a perspective view of the bioresorbable stent made in accordance with the teachings of the present invention.

FIG. 2 is an enlarged view of a partial segment of the bioresorbable stent made in accordance with the teachings of the present invention.

FIG. 3 is a side view of an alternate embodiment made in accordance with the teachings of the present invention.

FIG. 4 graphically depicts the bilateral self-expansion force of an alternate embodiment made in accordance with the teachings of the present invention versus UroLume® stents.

FIG. 5 graphically depicts the bilateral compression resistance of one embodiment made in accordance with the teachings of the present invention versus UroLume® stents.

FIG. 6 graphically depicts the radial self-expansion force by a Cuff Test of one embodiment made in accordance with the teachings of the present invention versus UroLume® stents.

FIG. 7 graphically depicts the radial compression resistance by a Cuff Test of one embodiment made in accordance with the teachings of the present invention versus UroLume® stents.

FIG. 8 graphically depicts the bilateral self-expansion force of one embodiment made in accordance with the teachings of the present invention as a function of in vitro aging time.

FIG. 9 graphically depicts the bilateral compression resistance of one embodiment made in accordance with the teachings of the present invention as a function of in vitro aging time.

FIG. 10 graphically depicts the radial compression resistance of an alternate embodiment made in accordance with the teachings of the present invention versus a UroLume® stent.

FIG. 11 graphically depicts the radial self-expansion force of an alternate embodiment made in accordance with the teachings of the present invention versus a UroLume® stent.

FIG. 12 graphically depicts the bilateral compression force versus calculated lumen area of bioresorbable stents made in accordance with the teachings of the present invention.

FIG. 13 graphically depicts the bilateral compression resistance as a function of time in vitro of various embodiments of bioresorbable fenestrated tube stents made in accordance with the teachings of the present invention.

FIG. 14 graphically depicts the bilateral self-expansion force as a function of time in vitro of various embodiments of bioresorbable tube stents made in accordance with the teachings of the present invention.

### DETAILED DESCRIPTION OF THE INVENTION

The present invention provides bioresorbable biocompatible stents, and methods for their production. In accordance with the teachings of the present invention, the bioresorbable stents of the present invention can be used in a wide variety applications that require controlled stent degradation over a period of time. In particular, it is contemplated that the bioresorbable stents of the present invention be used to alleviate urethral stenosis. Moreover, the bioresorbable stents of the present invention include novel braided patterns that provide large radial forces that maintain the patency of occluded regions. In another embodiment of the present invention, the stents provide openings that allow tissue in-growth (pseudopolypoid edematous tissue response) through the stents, thereby anchoring the stents in place and allowing the stents to be controllably degraded in the body without causing total stent collapse and obstruction. Furthermore, the present invention teaches an easy and cost effective method of producing the bioresorbable stents of the present invention while allowing for design flexibility. In particular, the present invention teaches methods of adjusting the in vivo functional life of bioresorbable stents through an annealing process.

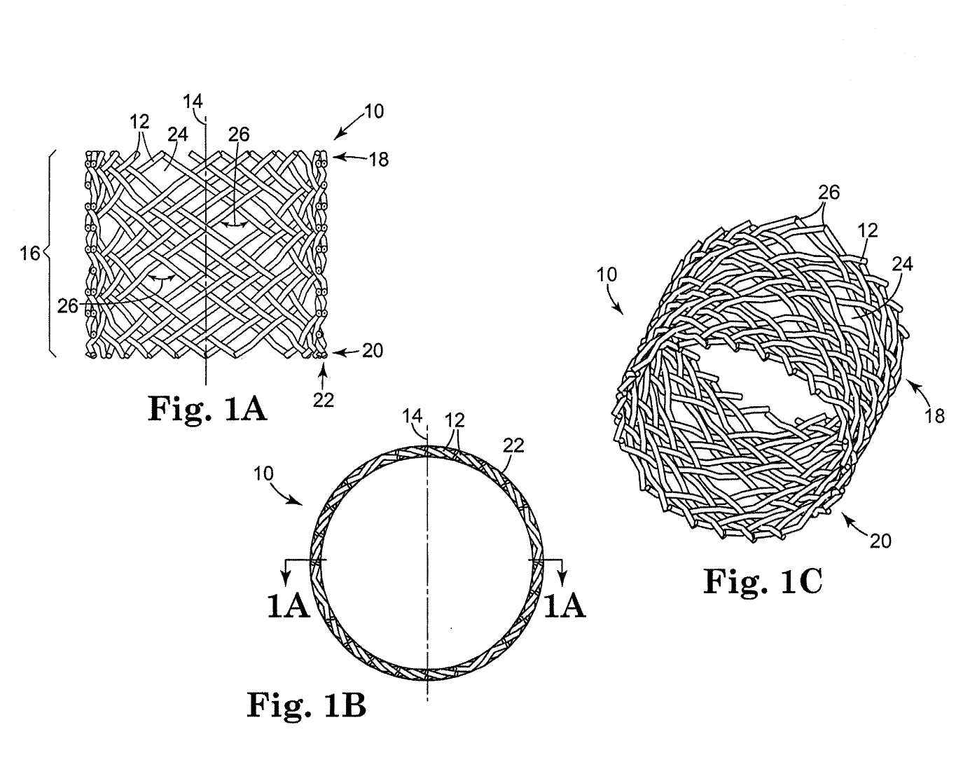

Referring more particularly to the figures, FIGS. 1A-1C illustrate the first embodiment of the bioresorbable, self-expanding stent 10 of the present invention. FIGS. 1A-1C show the bioresorbable stent 10 comprising a cylindrical sleeve having a first end 18 and a second end 20. A plurality of monofilaments 12 which are positioned substantially parallel and helically wound about the longitudinal axis 14 of the stent 10 form a latticed network 16. The latticed network 16 forms the wall 22 of the bioresorbable stent. As shown in FIGS. 1A-1C, the monofilaments 12 are braided in an alternating under-two-over-two pattern forming the latticed network. The braid-crossing angle 26 is the obtuse angle between any two monofilaments 12 at a point of intersection. In the first embodiment of the present invention, thirty to forty-eight monofilaments may be braided to form the bioresorbable stent 10; preferably forty monofilaments are braided to form the bioresorbable stent. The present invention also contemplates braiding patterns such as, but not limited to, under-one-over-one, under-one-over-two, under-one-over-three, under-two-over-three, under-three-over-three, and the like.

Because forty monofilaments are used on a 48 carrier braiding device, uneven openings result as shown in FIGS. 1A-1C. That is, the openings in the latticed network are not uniform. However, those skilled in the art will appreciate that uniform openings may be provided in a bioresorbable stent by manufacturing the stent on a braiding device with the appropriate number of evenly spaced carriers. For example, a thirty-strand stent may be formed on a 30 carrier braiding device. Uniform openings may also be achieved by pairing strands in a 48-strand stent with the under-two-over-two braid pattern.

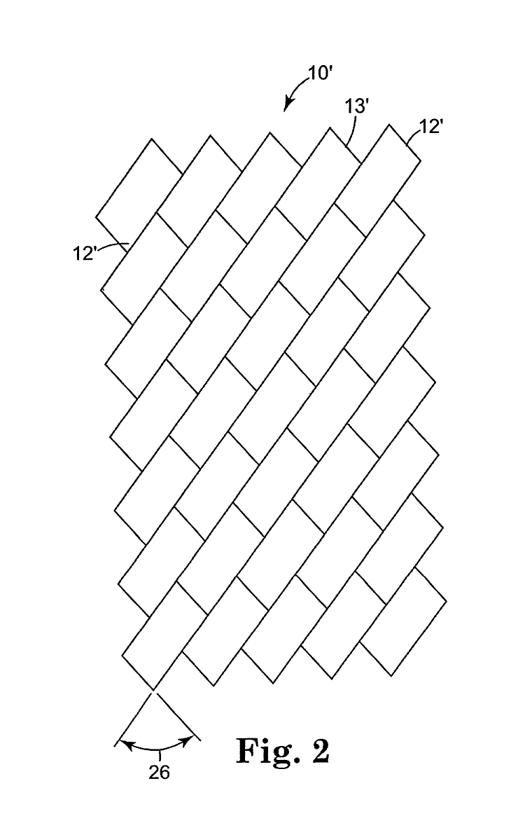

FIG. 2 is an enlarged view showing the under-two-over-two braiding pattern of the bioresorbable stents 10, 10′ of the present invention. Furthermore, FIG. 2 illustrates a bioresorbable stent 10′ having a single strand shift. A single strand shift is defined as adjacent monofilaments 12′, 13′ having a different braiding pattern. For instance, a monofilament 12′ will have an under-two-over-two braiding pattern and the adjacent monofilament 13′ will have an under-two-over-two braiding pattern offset by one monofilament. Stated differently, any adjacent monofilaments will not go “under and over” the same monofilaments.

FIGS. 1A-1C also show openings 24 between the individual monofilaments 12 that comprise the latticed network 16 of the stent 10. Providing spaces throughout the latticed network 16 of the stent 10 allows for sufficient tissue in-growth between the monofilaments of the latticed network thereby fixing the stent in position and minimizing the likelihood of stent misalignment or dislodgment. Those skilled in the art will appreciate that bioresorbable stents having openings of different sizes are also contemplated in the present invention provided that suitable self-expansion forces and compression resistance are achieved.

Those skilled in the art will also appreciate that the bioresorbable stents of the present invention may be made from a plurality of bioresorbable, biocompatible polymers. In a preferred embodiment of the bioresorbable stent (10, 10′), it is contemplated that the stent is comprised of monofilaments made from poly-L-lactic acid. It is also contemplated that the bioresorbable, biocompatible polymer may include, but is not limited to, polyanhydrides, polycaprolactones, polyglycolic acids, poly-L-lactic acids, poly-D-L-lactic acids, polydioxanone, and polyphosphate esters. Furthermore, it is contemplated that blends or copolymers of the aforementioned biocompatible polymers may be used to form the bioresorbable stents of the present invention. The different blends of polymers include, but are not limited to, those blends described and disclosed in co-pending U.S. patent application Ser. No. 09/324,743, the entire disclosure of which is hereby incorporated by reference.

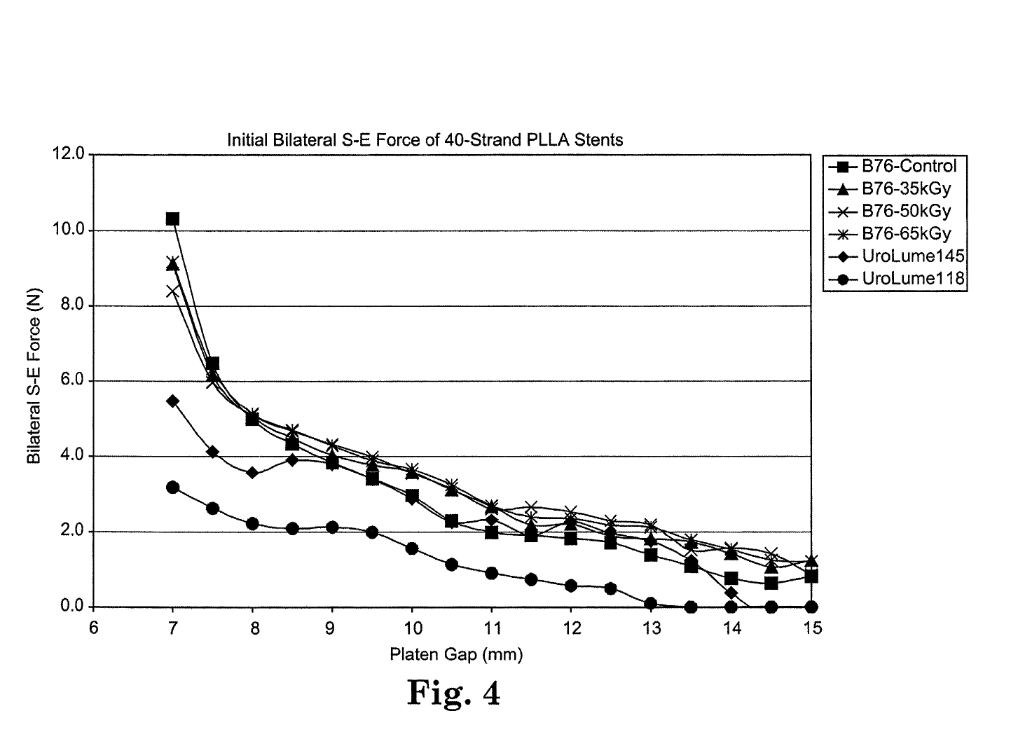

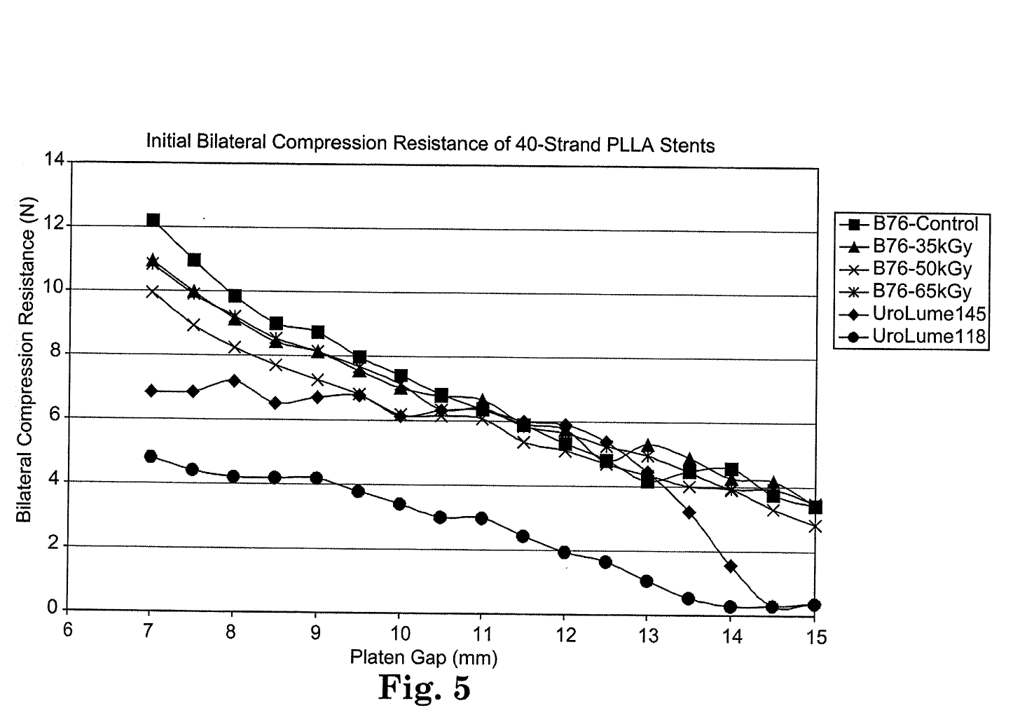

The under-two-over-two braided pattern as well as other braided patterns of the present invention are easy to manufacture, yet the braided patterns provide large radial forces as compared to traditional stents. FIGS. 4-5 graphically depict the bilateral self-expansion forces and compression resistance forces of one embodiment of the present invention versus UroLume® stents. UroLume® is the trademark for a metallic stent marketed by American Medical Systems, Inc., the assignee of the current application. In particular, FIGS. 4-5 graphically compare bioresorbable stents having 40 poly-L-lactic acid monofilaments braided in an under-two-over-two pattern and treated at various gamma irradiation doses (35 kGy, 50 kGy, and 65 kGy) versus UroLume® stents having braid-crossing angles of 118° and 145°.

The stent samples were subjected to a bilateral compression-relaxation test using an Instron test machine. The stents were compressed bilaterally between two smooth platens of a Delrin fixture from a resting state to a platen gap of 7 mm. The platen gap range of 7 mm to 15 mm corresponds to the stent diameter in a compressed state (7 mm) and an expanded state (15 mm). The stents were held for a set hold-time of approximately 1 minute, and the stents were allowed to relax. The stents were subjected to two cycles of compression, hold, and relaxation. The force exerted by the stent during the relaxation stage of the first cycle was recorded as the self-expansion force. The force applied to compress the stent in the second cycle was recorded as the compression resistance of the stent.

FIG. 4 illustrates that the bioresorbable stents of present invention have better bilateral self-expansion forces as compared to the UroLume® stents over a platen gap range of 7 mm to 15 mm. For instance, at a platen gap of 7 mm, a bioresorbable stent exposed to 35 kGy dose of gamma irradiation exerts a bilateral self-expansion force of approximately 9 N while UroLume® stents having braid-crossing angles of 118° or 145° exert self-expansion forces of 3N and approximately 5 N, respectively. FIG. 5 shows similar results were obtained when comparing the compression resistance of the bioresorbable stents with the UroLume stents® over a platen gap range of 7 mm to 15 mm. The bioresorbable stents exposed to 35 kGy, 50 kGy, and 65 kGy doses of gamma irradiation demonstrated greater bilateral compression resistance as compared to the UroLume® stents.

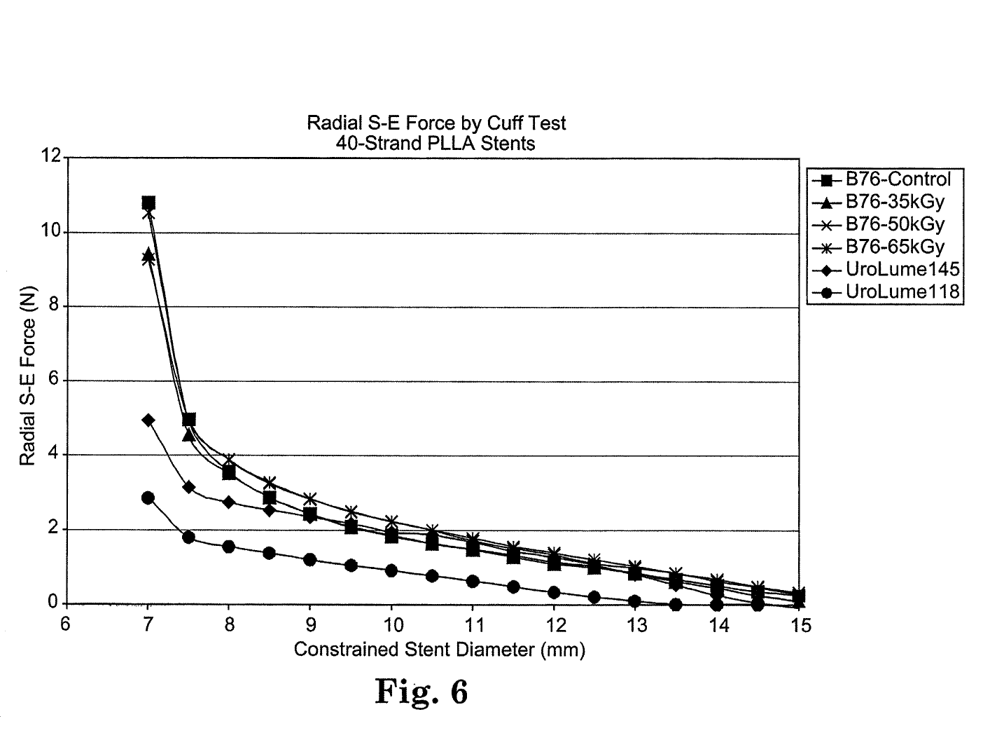

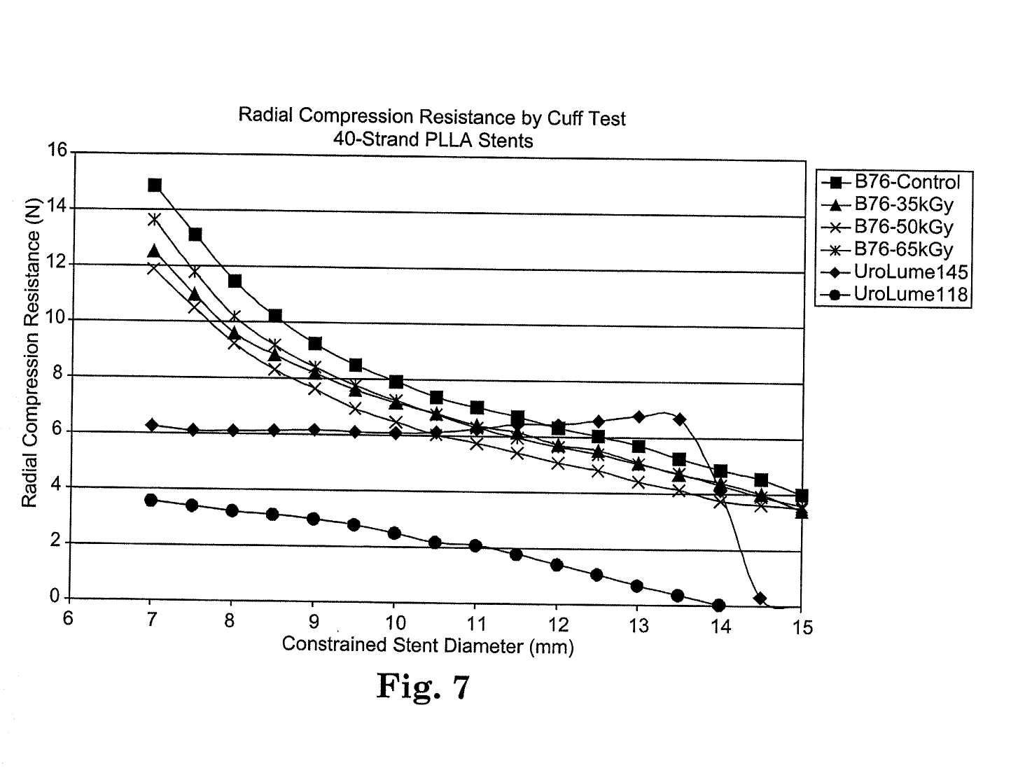

FIGS. 6-7 also show similar results when the stents of the present invention and UroLume® stents were subjected to a Cuff test. The Cuff test was conducted on an Instron test machine using a test fixture and a Mylare collar. The test fixture consists of a pair of freely rotating rollers separated by a 1-mm gap, and the Mylar® collar is a laminated film of Mylar® and aluminum foil. A 30-mm long stent segment was wrapped in a 25-mm wide collar and the two ends of the collar were passed together through the rollers of the test fixture. A pulling force was applied to the collar ends which radially compressed the stent against the rollers. The stent samples were compressed from their resting diameter to a predetermined diameter (typically 7-mm). The stent samples were compressed and held at the predetermined diameter for approximately one minute, and then they were allowed to relax. The stents were subjected to two cycles of compression, hold and relaxation. The force exerted by the stent during the relaxation stage of the first cycle was recorded as the self-expansion force. The force applied to compress the stent in the second cycle was recorded as the compression resistance of the stent.

The bioresorbable stents of the present invention demonstrated greater radial self-expansion forces over the whole range of constrained stent diameters from 7 mm to 15 mm as compared to the UroLume® stents. In particular, the bioresorbable stents displayed approximately 9 N to 11 N of radial self-expansion force at a constrained stent diameter of 7 mm as compared to 3 N and 5 N at 7 mm of radial self-expansion force for the UroLume stents, as shown in FIG. 6. The superior results are also illustrated by the graphical data in FIG. 7.

The graphical data set forth in FIGS. 4-7 illustrate that the bioresorbable stents having an under-two-over-two braided pattern have superior radial self-expanding forces and compression resistance forces as compared to UroLume® metallic stents. Furthermore, the bioresorbable stents of the present invention are also controllably biodegradable which eliminates the need for complicated or invasive stent removal procedures. That is, once an implanted stent has served its intended function, the stent is controllably degraded and naturally eliminated by the human body.

The bioresorbable, self-expanding stents are manufactured by providing a plurality of monofilaments and braiding these monofilaments in an under-two-over two pattern to form a latticed network as shown in FIG. 1 and FIG. 2. As previously stated, it is contemplated that the latticed network of the bioresorbable stents comprises thirty to forty-eight monofilaments. The latticed network is formed by winding the monofilaments about a mandrel. Approximately hall of the monofilaments are wound around the mandrel in a clockwise direction while the other half of the monofilaments are wound in a counter-clockwise direction. The angle between the two filaments at the point where they intersect is defined as the braid-crossing angle 26 as shown in FIG. 1. It is contemplated that the monofilaments intersect at a braid-crossing angle between 100° to 150°. In a preferred embodiment, the bioresorbable stents comprise monofilaments having an as-braided braid-crossing angle of 110°. Those skilled in the art will appreciate that other braid-crossing angles may be selected to achieve different self-expansion forces or compression resistance.

The bioresorbable stents then undergo an annealing process. The annealing process includes placing the bioresorable stents on a mandrel, axially compressing the stents by 30% to 60%, heating the stents to the glass transition temperature of the biocompatible polymer for a predetermined period of time, and allowing the stents to be controllably cooled. The annealing process relieves internal stresses and instabilities of the monofilaments that result from the production of the bioresorbable stents. In a preferred embodiment of the present invention where the latticed structure is formed from poly-L-lactide monofilaments, the bioresorbable stents are heated to approximately 90° C. for a length of time between about one and about eight hours, preferably four hours, in an inert atmosphere. The inert atmosphere may be comprised of a high vacuum or nitrogen gas. Those skilled in the art will appreciate that other inert atmospheres having low moisture content are also contemplated including, but not limited to, argon, or helium. The bioresorbable stents are then controllably cooled to room temperature. Each stent is then cut to desired size for its intended application. Thereafter, the stents are exposed to Co⁶⁰ gamma irradiation to fine tune the in vivo functional life of the bioresorbable stents. Exposure to gamma irradiation causes molecular degradation of the polymers that comprise the bioresorbable stents; however, the gamma irradiation does not affect the overall morphology of the polymers.

During the annealing process, the monofilaments that comprise the bioresorbable stent contract resulting in a different final braid-crossing angle. In contrast to traditional methods where the monofilaments are annealed prior to braiding, the contraction of the monofilaments that comprise the braided stent is important in achieving the compression resistance and self-expansion forces for the stents of the present invention. The final post-annealing braid angle ranges from approximately 125° to 150°, and more particularly a final braid angle ranging from approximately 130° to 145°. Those skilled in the art will appreciate that the final post-annealing braid angle is dependent upon the desired properties and stent length. For instance, a 1.5 cm long stent would require a final post-annealing braid angle ranging from approximately 139° to 145° whereas a lesser braiding angle might be adequate for a longer stent.

The in vivo functional life of the bioresorbable stents is related to the temperature and duration of the annealing process and the dosage of gamma irradiation. Accordingly, the functional lifetime of the stents can be controlled and/or adjusted by manipulating the annealing conditions during the manufacturing process. In one embodiment of the present invention, the annealing conditions of 90° C. for a length of time between about one to about eight hours, preferably four hours, in an inert atmosphere followed by 50 kGy dose of gamma irradiation provides bioresorbable stents having approximately a two week functional life and substantial stent degradation by approximately the fourth week of in vivo implantation. In another embodiment of the present invention, the bioresorbable stents may be annealed at a temperature higher than 110° C. for at least eight hours to achieve an in vivo functional life between three to six months. The bioresorbable stents are typically annealed at 110° C. for approximately eighteen hours to achieve an in vivo functional life between three to six months. Those skilled in the art will appreciate that the annealing parameters mal be adjusted for shorter or longer in vivo functional lives.

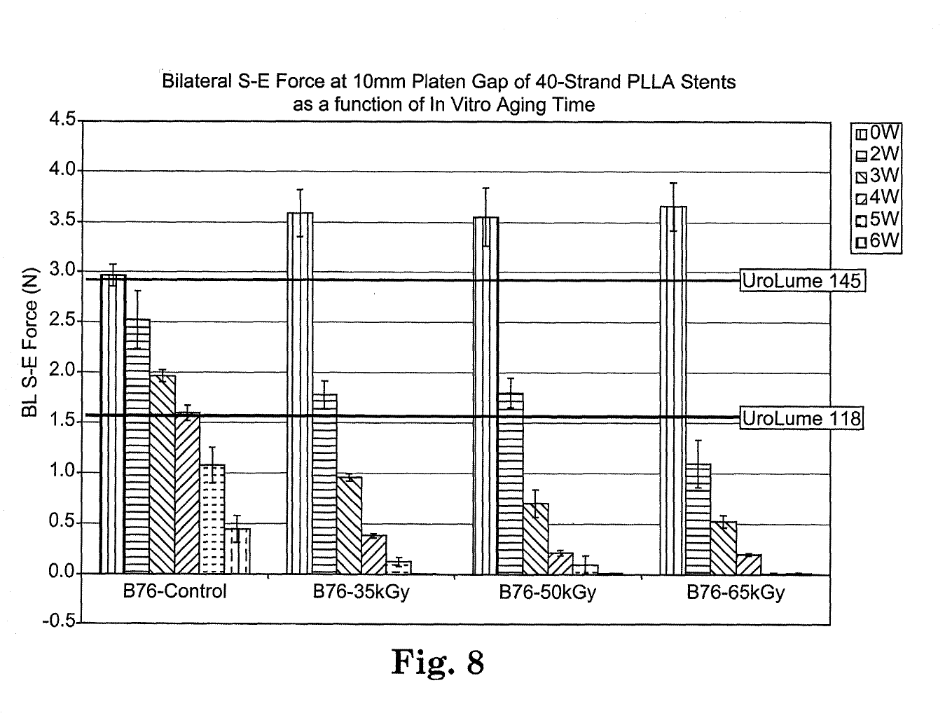

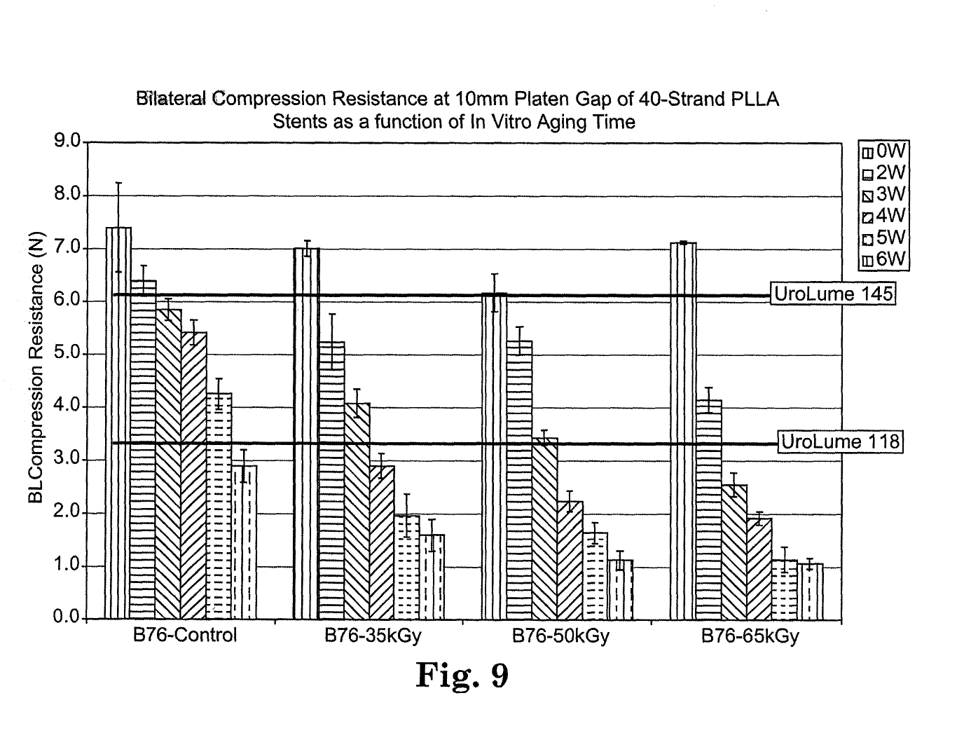

FIGS. 8-9 graphically illustrate the mechanical strengths of the bioresorbable stents of the present invention as a function of in vitro aging time. The in vitro study parameters were designed to mimic in vivo functional life. Accordingly, the stents were aged in a phosphate buffered saline (pH 7.3) at 37° C., and samples were then tested in a bilateral compression/relaxation test at each corresponding aging period. In particular, FIGS. 8-9 show the changes in the self-expansion force and bilateral compression resistance of the bioresorbable stents over a six week period of time. For instance, as shown in FIGS. 8-9, the stents exposed to 35 kGy and 50 kGy doses of gamma irradiation retained ≧70% of their initial mechanical strength for two weeks, but a substantial degradation in mechanical strength had occurred by the fourth week.

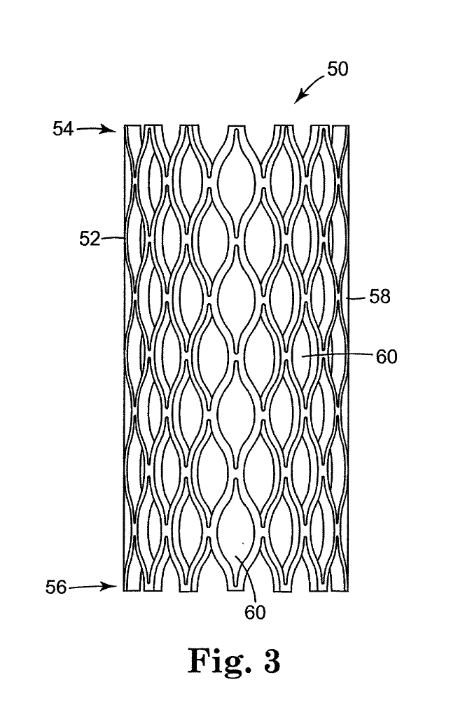

FIG. 3 illustrates a second embodiment of the present invention. The second embodiment of the present invention is similar to the laser cut stent as disclosed in U.S. Pat. No. 5,356,423, the entire contents which are herein incorporated by reference. The bioresorbable stent 50 is comprised of a tubular sheath 52 having a first end 54 and a second end 56. A walled surface 58 having a plurality of fenestrations 60 spaced throughout the walled surface 58 is shown in FIG. 3. The walled surface 58 is contemplated to have a thickness of 0.025″ to 0.030″, preferably 0.030″. The fenestrations 60 are shaped in such a mariner to maximize the number of openings for tissue in-growth while maintaining the predetermined self-expansion and compression resistance forces of the bioresorbable stent.

The bioresorbable stents, as shown in FIG. 3, are formed by the following process. Bioresorbable, biocompatible polymers are injection molded or extruded into a tubular sheath. The polymers may be selected from any known bioresorbable polymers including, but not limited to, polyanhydrides, polycaprolactones, polyglycolic acids, poly-L-lactic acids, poly-D-L-lactic acids, polydioxanone, and polyphosphate esters. In a preferred embodiment, polydioxanone is used to form the tubular sheath. Furthermore, it is contemplated that blends or copolymers of the aforementioned biocompatible polymers may be used to form the bioresorbable stents of the present invention. The tubular sheath may be injection molded with or without fenestrations. In a preferred method, the tubular sheath is injection molded without fenestrations. The fenestrations are introduced into the tubular sheaths by cutting processes including, but not limited to, laser cutting and machining.

The bioresorbable stents then undergo an annealing process. The annealing process includes heating the stents to or above the glass transition temperature of the biocompatible polymer for a predetermined period of time, and allowing the stents to cool slowly. The annealing process relieves internal stresses and instabilities that result from the production of the bioresorbable stents of the present invention. Bioresorbable stents made from polydioxanone are heated to a temperature of approximately 75° C. for between about one and six hours, preferably three hours, in an inert atmosphere of high vacuum or nitrogen gas and controllably cooled for approximately twelve hours. Those skilled in the art will appreciate that other inert atmospheres having low moisture content are also contemplated including, but not limited to, argon, or helium.

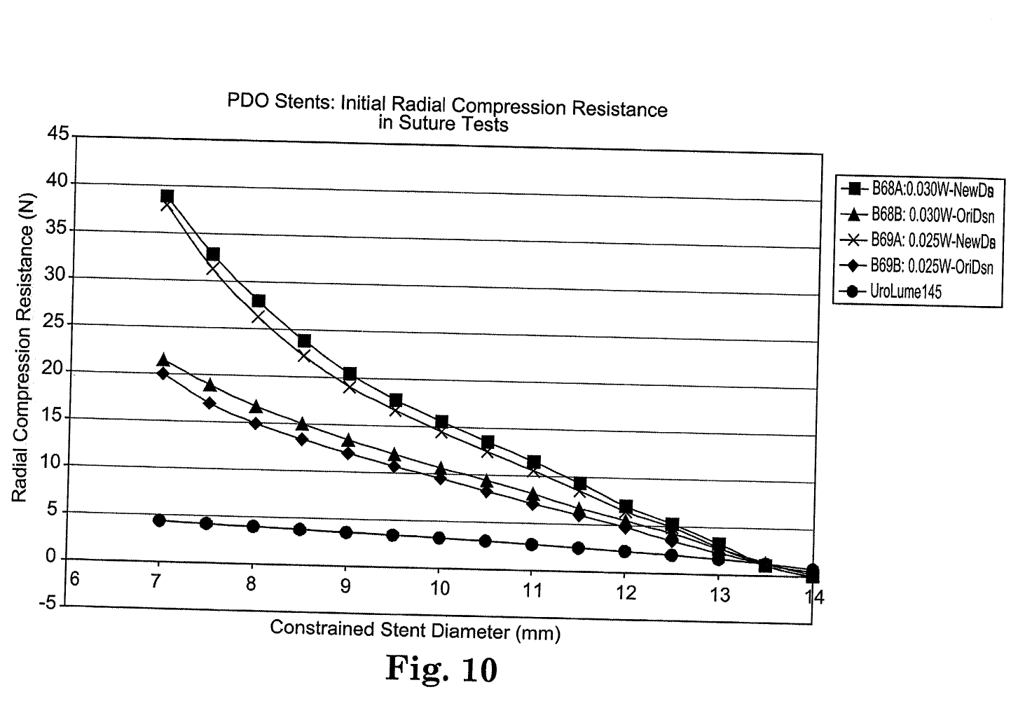

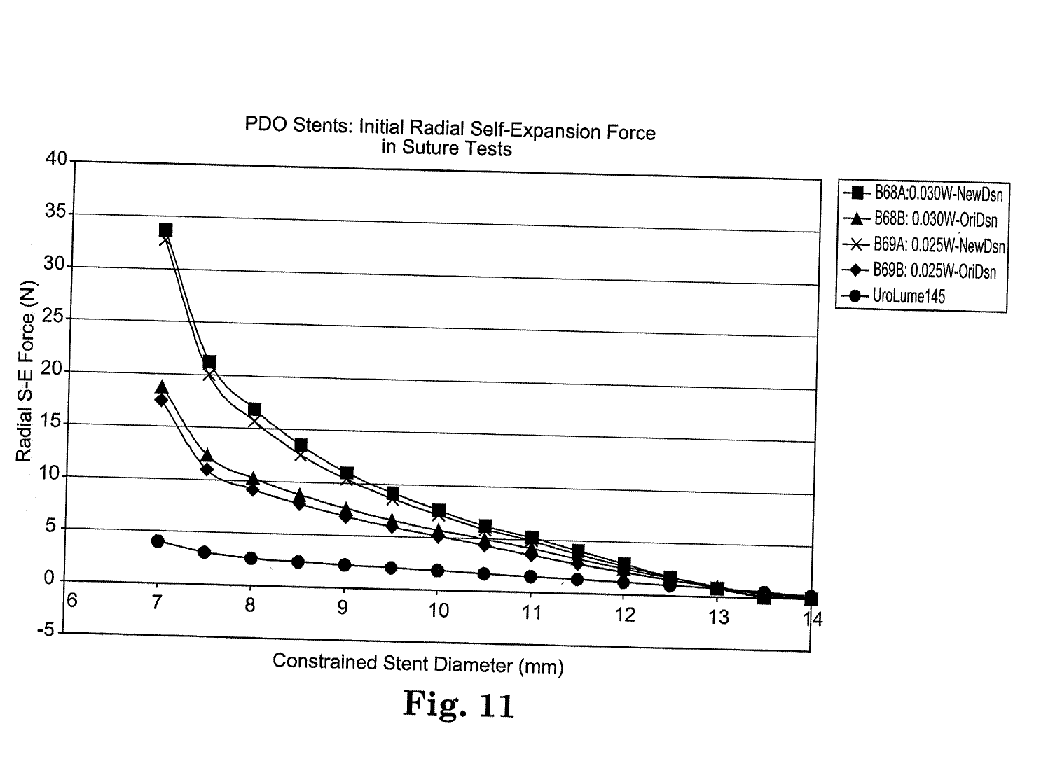

The graphical data set forth in FIGS. 10-12 illustrate the mechanical properties of the bioresorbable stent 50. In particular, FIGS. 10-11 graphically depict the radial compression resistance and self-expansion forces of two embodiments of the bioresorbable stent 50 having different fenestration designs and wall thickness versus a 145° UroLume® stent. The stent samples were subjected to a Suture test using an Instron test machine. The Suture test is similar to the Cuff test with the exception that a suture, rather than a Mylar® collar, is used to apply radial compression to the stent and the two ends of the suture are passed through a Delrin guide before passing through the rollers of the test fixture. Like the Cuff test, the stent samples were compressed and held at the predetermined diameter for approximately one minute, and then they were allowed to relax. The stents were subjected to two cycles of compression, hold and relaxation. The force exerted by the stent during the relaxation stage of the first cycle was recorded as the self-expansion force. The force applied to compress the stent in the second cycle was recorded as the compression resistance of the stent.

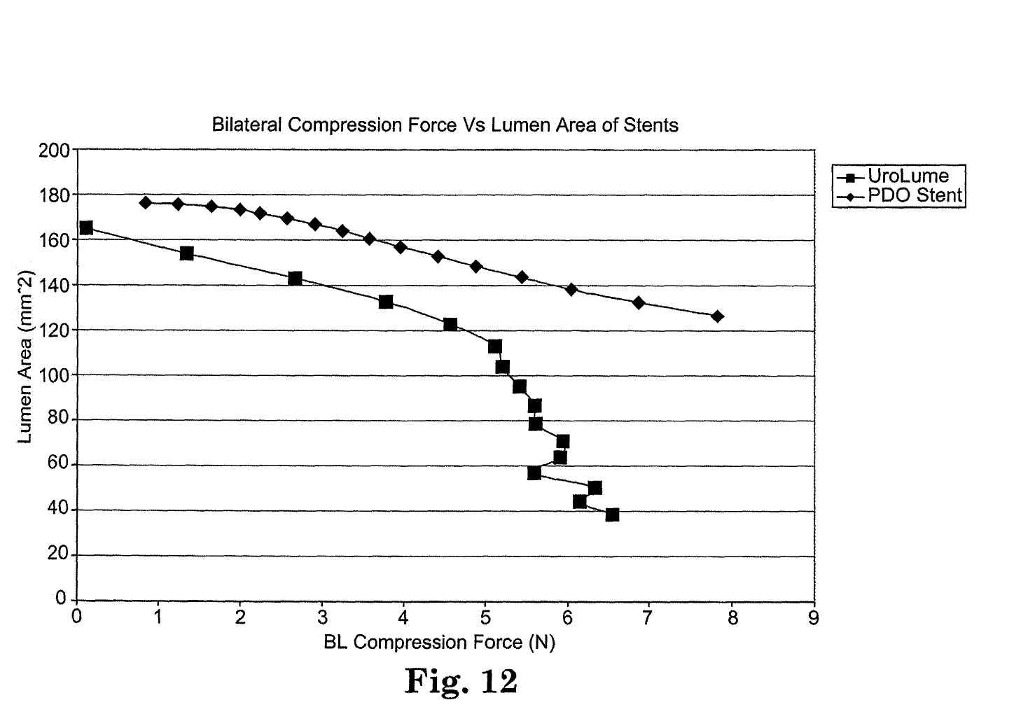

As shown in FIGS. 10-11, the bioresorbable stents of the present invention displayed substantially higher radial mechanical properties as compared to the UroLume® stent. FIG. 12 graphically depicts the cross-sectional lumenal area as a function of bilateral compression force for bioresorbable fenestrated tube stents and 145° UroLume® stent. FIG. 12 shows that for the same amount of bilateral compression, the reduction in the lumen size of a UroLume® metallic stent was significantly greater than that of the bioresorbable stent 50 of the present invention.

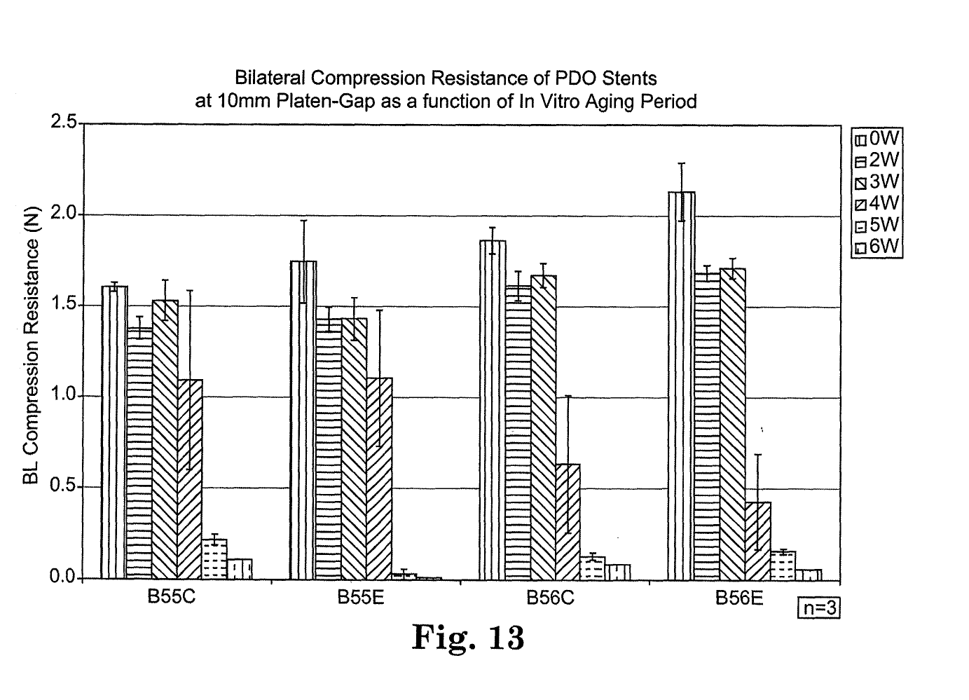

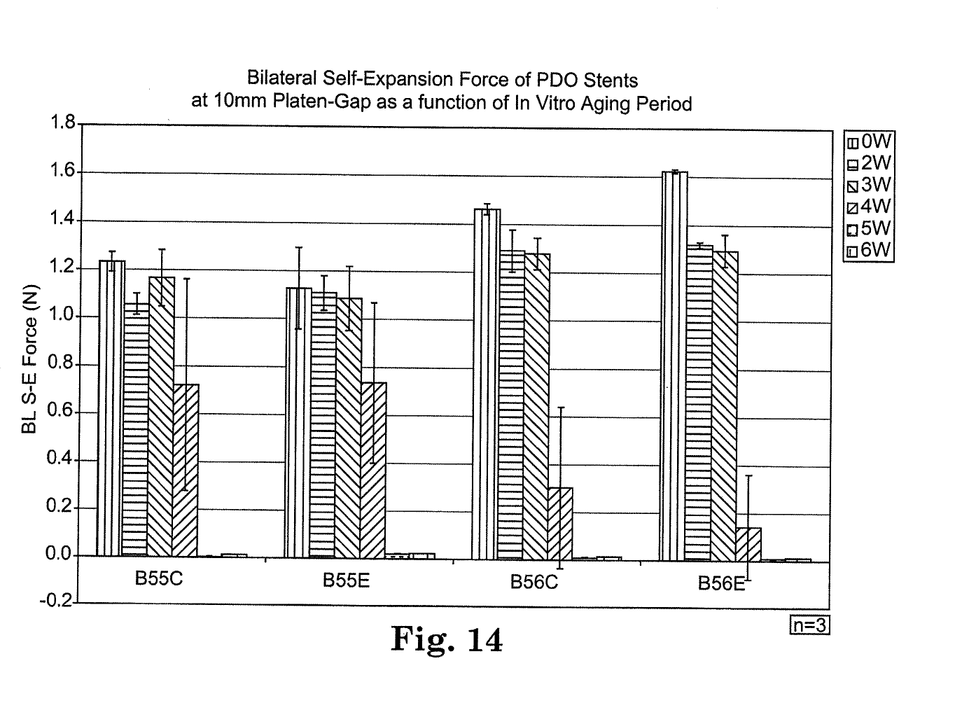

FIGS. 13 and 14 are bar charts that illustrate the compression resistance and self-expansion force as a function of in vitro aging for four bioresorbable fenestrated tube stents. The four test groups were subjected to different combinations of annealing and sterilization. Table 1 identifies the particular treatments that each test group received. The four test groups were aged in a phosphate buffered saline (pH 7.3) at 37° C., and samples were then subjected to a bilateral compression relaxation test at each aging period. FIGS. 13 and 14 show that all four test groups maintained approximately 80% to 95% of initial compression resistance and 88% to 100% of self-expansion force after three weeks of aging. Additionally, FIGS. 13 and 14 show that the annealed stents had approximately 18% to 23% higher initial compression resistance and approximately 25% to 45% higher initial self-expansion force than non-annealed stents. FIGS. 13 and 14 also show that ethylene oxide (EtO) sterilization provides some slightly increased mechanical properties. The data as shown in FIGS. 13 and 14 illustrate bioresorbable stents 50 that have a functional life of approximately two to four weeks.

TABLE 1
Test Groups used for In Vitro Strength Retention Study

| Test-Group ID | Annealing | Sterilization |
|---|---|---|
| B55C | None | None |
| B55E | None | EtO |
| B56C | Annealed | None |
| B56E | Annealed | EtO |

Bioresorbable stents made in accordance with the teachings of the present invention may be inserted into a constricted region of a body lumen by the following method. The stent is compressed and loaded into a delivery system. Once the delivery system is properly positioned in the constricted lumen, the stent is deployed and allowed to self-expand. While the stent is self-expanding, the stent concomitantly exerts a radial force against the walls of the lumen, thereby restoring the patency of the occluded region. The stents of the present invention are formed from bioresorbable polymers that provide sufficient radial strength to relieve stenosis. Additionally, bioresorbable stents having various predetermined lifetimes may be made in accordance with the present invention. Over a period of time the bioresorbable stents degrade and the body will excrete or absorb and metabolize the degradation product(s), thereby dispensing with complicated removal procedures.

In closing, it is to be understood that the embodiments of the invention disclosed herein are illustrative of the principles of the present invention. Other modifications that may be employed are within the scope of the invention. Thus, by way of example, but not of limitation, alternative configurations of the bioresorbable, self-expanding stent may be utilized in the treatment of urethral stenoses. Accordingly, the present invention is not limited to that precisely as shown and described in the present invention.

The terms “a” and “an” and “the” and similar referents used in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventors expect skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A bioresorbable, self-expanding stent comprising:
a cylindrical sleeve having a first end and a second end;
a latticed network disposed between said first end and said second end of said cylindrical sleeve;
said latticed network formed from a plurality of monofilaments, wherein at least two of said monofilaments are braided in an alternating braid pattern; and
said plurality of monofilaments comprises at least one biocompatible polymer, and said cylindrical sleeve having a controllable in vivo lifetime.

2. The bioresorbable, self-expanding stent of claim 1 wherein said plurality of monofilaments ranges from 30 to 48 monofilaments.

3. The bioresorbable, self-expanding stent of claim 2 wherein said plurality of braided monofilaments comprise 40 monofilaments.

4. The bioresorbable, self-expanding stent of claim 1 further including at least a single strand shift between each adjacent monofilament.

5. The bioresorbable, self-expanding stent of claim 1 further including an as-braided braid-crossing angle ranging from approximately 100° to 150°.

6. The bioresorbable, self-expanding stent of claim 1 further including an as-braided braid-crossing angle of approximately 110°.

7. The bioresorbable, self-expanding stent of claim 1 further including a post-annealed braid-crossing angle ranging from approximately 125° to 150°.

8. The bioresorbable, self-expanding stent of claim 1, wherein said braid pattern is selected from the group consisting of under-one-over-one, under-one-over-two, under-one-over-three, under-two-over-two, under-two-over-three, and under-three-over-three.

9. A bioresorbable, self-expanding stent comprising:
a cylindrical sleeve having a first end and a second end;
a latticed network disposed between said first end and said second end of said cylindrical sleeve;
said latticed network formed from a plurality of monofilaments helically wound about a longitudinal axis of said cylindrical sleeve, wherein approximately one-half of said plurality of monofilaments are wound in a clockwise direction and approximately one-half of said plurality of monofilaments are wound in a counter-clockwise direction, and said plurality of monofilaments are braided in an alternating braid pattern; and
said plurality of braided monofilaments comprises at least one biocompatible polymer, and said cylindrical sleeve having a controllable in vivo lifetime.

10. The bioresorbable, self-expanding stent of claim 9 wherein said plurality of monofilaments ranges from 30 to 48 monofilaments.

11. The bioresorbable, self-expanding stent of claim 10 wherein said plurality of braided monofilaments comprise 40 monofilaments.

12. The bioresorbable, self-expanding stent of claim 9 further including a single strand shift between each adjacent monofilament.

13. The bioresorbable, self-expanding stent of claim 9 further including an as-braided braid-crossing angles ranging from approximately 100° to 150°.

14. The bioresorbable, self-expanding stent of claim 9 further including an as-braided braid-crossing angle of approximately 110°.

15. The bioresorbable, self-expanding stent of claim 9 further including an post-annealed braid-crossing angle ranging from approximately 125° to 150°.

16. The bioresorbable, self-expanding stent of claim 9, wherein said braid pattern is selected from the group consisting of under-one-over-one, under-one-over-two, under-one-over-three, under-two-over-two, under-two-over-three, and under-three-over-three.

17. A bioresorbable, self-expanding stent comprising:
a cylindrical sleeve having a first end and a second end;
a latticed network disposed between said first end and said second end of said cylindrical sleeve;
said latticed network formed from approximately forty monofilaments helically wound about a longitudinal axis of said cylindrical sleeve, wherein approximately fifteen to twenty of said monofilaments are wound in a clockwise direction and approximately fifteen to twenty of said monofilaments are wound in a counter-clockwise direction, wherein said approximately thirty to forty monofilaments are braided in an alternating braid pattern, wherein said braid pattern is selected from the group consisting of under-one-over-one, under-one-over-two, under-one-over-three, under-two-over-two, under-two-over-three, and under-three-over-three.; and
said plurality of braided monofilaments comprises poly-L-lactide polymers, and said cylindrical sleeve having a controllable in vivo lifetime.

18. A bioresorbable, self-expanding stent comprising:
a tubular sheath having a first end and a second end; and
a fenestrated walled surface disposed between said first end and said second end, said fenestrated walled surface comprised of at least one biocompatible polymer, and said fenestrated walled surface having a controllable in vivo lifetime.

19. The bioresorbable, self-expanding stent of claim 18 wherein said at least one biocompatible polymer is polydioxanone.

20. The bioresorbable, self-expanding stent of claim 18 wherein said tubular sheath has an inner diameter ranging from 12 mm to 18 mm.

21. The bioresorbable, self-expanding stent of claim 18 wherein said tubular sheath has an inner diameter of approximately 15 mm.

22. A bioresorbable, self-expanding stent comprising:
a tubular sheath having a first end and a second end, said tubular sheath having an inner diameter ranging from 12 mm to 18 mm; and
a fenestrated walled surface disposed between said first end and said second end, said fenestrated walled surface comprised of at least one biocompatible polymer, and said fenestrated walled surface having a controllable in vivo lifetime.

22. The bioresorbable, self-expanding stent of claim 22 wherein said tubular sheath has an inner diameter of approximately 15 mm.

23. The bioresorbable, self-expanding stent of claim 22 wherein said at least one biocompatible polymer is polydioxanone.

24. bioresorbable, self-expanding stent comprising:
a tubular sheath having a first end and a second end, said tubular sheath having an inner diameter of approximately 15 mm; and
a fenestrated walled surface disposed between said first end and said second end, said fenestrated walled surface comprised of polydixanone, wherein said tubular sheath has a controllable in vivo lifetime.

25. A method of producing a bioresorbable, self-expanding stent comprising:
providing biocompatible, bioresorbable monofilaments;
braiding said monofilaments into a latticed network, said latticed network having an alternating braiding pattern; and
annealing said latticed structure.

26. The method of claim 25 further comprising:
adjusting annealing conditions to achieve a predetermined in vivo functional life.

27. The method according to claim 25 wherein said biocompatible, bioresorbable monofilaments are poly-L-lactide monofilaments.

28. The method according to claim 25 wherein said annealing step further includes heating said latticed structure to 90° C. in an inert atmosphere.

29. The method according to claim 28 wherein said inert atmosphere is selected from the group consisting of nitrogen, argon, and helium.

30. The method according to claim 28 wherein said inert atmosphere comprises a high vacuum.

31. The method according to claim 25 further comprising:
axially compressing said latticed structure by 30% to 60% prior to said annealing step.

32. The method of claim 25 further comprising:
exposing said annealed latticed structure to gamma irradiation.

33. The method according to claim 32 wherein said latticed structure is exposed to approximately 35 kGy to 75 kGy total dose of gamma irradiation.

34. A method of producing a bioresorbable, self-expanding stent comprising:
providing biocompatible, bioresorbable monofilaments;
braiding said biocompatible, bioresorbable monofilaments into a latticed structure, wherein said biocompatible, bioresorbable monofilaments are woven in an alternating braiding pattern; and
annealing said latticed structure at approximately 90° C. in an inert atmosphere wherein said inert atmosphere is selected from the group consisting of nitrogen, argon, helium, and high vacuum.

35. The method according to claim 34 further comprising:
axially compressing said latticed structure on a mandrel by 30% to 60% prior to said annealing step.

36. The method according to claim 34 further comprising:
exposing said annealed latticed structure to gamma irradiation.

37. The method according to claim 36 wherein said latticed structure is exposed to approximately 35 kGy to 75 kGy total dose of gamma irradiation.

38. A method of producing a bioresorbable, self-expanding stent comprising:
(a) providing poly-L-lactide monofilaments;
(b) braiding said poly-L-lactide monofilaments into a latticed structure, wherein said poly-L-lactide monofilaments are woven in an alternating under-two-over-two pattern;
(c) axially compressing said latticed structure on a mandrel by 30% to 60%;
(d) annealing said latticed structure at approximately 90° C. for at least one hour in an inert atmosphere, wherein said inert atmosphere is selected from the group consisting of nitrogen, argon, helium, and high vacuum; and
(e) exposing said latticed structure to approximately 35 kGy to 75 kGy total dose of gamma irradiation.

39. A method of producing a stent comprising:
selecting a biocompatible, bioresorbable polymer;
forming a tubular sheath having fenestrations from said biocompatible, bioresorbable polymer; and
annealing said tubular sheath.

40. The method according to claim 39 wherein said forming step further comprises injection molding or extruding said tubular sheath.

41. The method according to 39 wherein said annealing step further comprises heating said tubular sheath to a temperature of approximately 75° C. for approximately one to three hours.

42. The method according to claim 41 wherein said annealing step further includes exposing said tubular sheath to an inert atmosphere inert atmosphere is selected from the group consisting of nitrogen, argon, and helium.

43. The method according to claim 41 wherein said annealing step further includes exposing said tubular sheath to a high vacuum.

44. The method according to claim 39 wherein said forming step further comprises laser cutting said fenestrations.

45. A method of producing a stent comprising:
selecting a biocompatible, bioresorbable polymer;
forming a tubular sheath from a biocompatible, bioresorbable polymer;
cutting fenestrations into said tubular sheath; and
annealing said tubular sheath to a temperature of approximately 75° C. for approximately one to three hours in an inert atmosphere.

46. The method according to claim 45 wherein said annealing step further includes exposing said tubular sheath to nitrogen.

47. The method according to claim 45 wherein said annealing step further includes exposing said tubular sheath to high vacuum.

48. A method of producing a stent comprising:
providing polydioxanone polymers;
injection molding a tubular sheath from said polydioxanone polymers;
laser cutting fenestrations into said tubular sheath; and
annealing said tubular sheath at a temperature of approximately 75° C. for at least one hour in an inert atmosphere of high vacuum or nitrogen gas.

49. A bioresorbable, self-expanding stent comprising:
a cylindrical sleeve having a first end and a second end;
a latticed network disposed between said first end and said second end of said cylindrical sleeve;
said latticed network formed from approximately forty monofilaments helically wound about a longitudinal axis of said cylindrical sleeve, wherein approximately twenty of said monofilaments are wound in a clockwise direction and approximately twenty said monofilaments are wound in a counter-clockwise direction, wherein said approximately forty monofilaments are braided in an alternating under-two-over-two braid pattern; and
said plurality of braided monofilaments comprises poly-L-lactide polymers, and said bioresorbable stent having a controllable in vivo lifetime of at least two weeks.

50. A method for using a bioresorbable, self-expanding stent comprising:
disposing said bioresorbable, self-expanding stent in a delivery system, said bioresorbable, self-expanding stent having a controlled in vivo lifetime;
inserting said delivery system into a constricted region within a body canal;
deploying said bioresorable stent into said constricted region; and
allowing said bioresorbable stent to self-expand and restore patency of said constricted region.
